(19) United States
(12) Patent Application Publication
(10) Pub. No.: US 2023/0162871 A1
(43) Pub. Date: May 25, 2023

(54) CARE LIFECYCLE TELE-HEALTH SYSTEM AND METHODS

(71) Applicant: VeeOne Health, Roseville, CA (US)

(72) Inventors: Arshad Ali, Roseville, CA (US); Ijaz Artif, Roseville, CA (US); Abdul Rahim Khatri, San Ramon, CA (US)

(21) Appl. No.: 18/057,388

(22) Filed: Nov. 21, 2022

Related U.S. Application Data
(60) Provisional application No. 63/264,423, filed on Nov. 22, 2021.

(51) Int. Cl. G16H 80/00; G16H 40/20; G16H 10/60

(52) U.S. Cl. CPC G16H 80/00; G16H 10/60; G16H 40/20

(57) ABSTRACT

A tele-health system comprising a tele-health subsystem further comprising a plurality of portals having a plurality of functionalities; wherein an individual portal from the plurality is unique to a particular actor in a continuum of care, the portal further comprising visual, auditory, textual and derived data presented in a controlled environment to enable a tele-health session; a patient located tele-health subsystem further comprising audio and video interchange between at least two locations; wherein the audio and video interchange is a treatment episode related to a patient generated at the patient portal; a consultant located tele-health subsystem further comprising audio and video interchange between at least two locations; wherein the audio and video interchange is a treatment episode related to a patient presented via a consultant portal; and an administration tele-health subsystem further comprising an administration of a tele-health episode related to a patient that manages a set of all related data collected and presented regarding a particular patient during care and further comprising a data update function that adds a set of new data upon new information about the patient generated by at least one of the consultants.

### CROSS-REFERENCE TO RELATED APPLICATIONS

This application claims the benefit of U.S. Provisional Application No. 63/264,423, filed November 22. 2021, entitled CARE LIFECYCLE TELE-HEALTH SYSTEM AND METHODS, the entirety of which is hereby incorporated by reference.

### CARE LIFECYCLE TELE-HEALTH SYSTEM AND METHODS

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the drawings, that form a part of this document: Copyright 2021, VeeOne Health, Inc.. All Rights Reserved

### TECHNICAL FIELD

This document pertains generally, but not by way of limitation, to optimizing care, assessment and treatment of individuals via telehealth technology

### BACKGROUND

Telemedicine has a variety of applications in patient care, education, research, administration, and public health. Some uses such as emergency calls to 911 numbers using ordinary telephones are so commonplace that they are often overlooked as examples of distance medicine. Other applications such as tele-surgery involve technologies and procedures that are still in the experimental stage. The use of interactive video for such varied purposes as psychiatric consultations and home monitoring of patients attracts much attention and news coverage, although such applications are far from routine in everyday medical practice.

For many decision makers, the case for new or continued investment in telemedicine remains incomplete, particularly given the competition for resources in an era of budgetary retrenchment in health care and government. Most clinical applications of telemedicine have not been subjected to systematic comparative studies that assess their effects on the quality, accessibility, or cost of health care. Although telemedicine is hardly unique among health care services in lacking evidence of its effectiveness, the increasing demand for such evidence by health plans, patients, clinicians, and policymakers challenges advocates of clinical telemedicine to undertake more and better evaluations of its practicality, value, and affordability.

The first problem with tele-health systems identified by the inventors is the lack of connectivity to many of the surrounding systems

One of the problems identified by the inventors is that traditional hospital information modules (HIM) do not deal well with the rubric of the tele-health work stream namely integrating with a number of tele-health and electronic healthy records (EHR) products while maintaining compliance with HIPAA HEALTH INSURANCE PORTABILITY AND ACCOUNTABILITY ACT OF 1996 (HIPAA) and other privacy regimes that indirectly impact data sets related to PHI (Personals Health Information)

A second problem identified with respect to tele-health-HIM systems is that alerts that would be seen in a large number of internal systems at hospital are not seen by a tele-health consultant or in many cases do not exist in an ambulatory HIM. These missing or mistimed alerts can significantly alter a treatment regime if missed from a tele-health diagnosis.

A third problem identified with respect to the tele-health HIM is missing data from the various EHR sources that would normally be input by an attending nursing staff or medical assistant. This in many cases is missed as the patient in some cases is out in the field being tended to by an EMT or in a smaller care facility where the respective attendants lack the training or the focus to input data correctly.

Another problem identified is that tele-health takes the patient in a number of locations where full diagnostic or EHR data is missing for the tele-health HIM usage. Thus a problem of how to fill in or estimate missing data is also a substantial problem with the tele-health expert being shown a scene from an accident, a battlefield, an ambulance or other non-clinical settings presents itself to the tele-health expert.

Another problem identified is that consultants maybe in remote sites as well limited by bandwidth or connectivity and a tele-health system struggles to provide the right information at the right time

Another problem identified is that patients and consultants may both be in transitory or ambulatory conditions and a tele-health system struggles to adapt to this right information at the right times

Another problem identified is the ability of patient handoff and coverage between care providers, ambulance services, EMTs and to successfully transfer all related matters between systems to from initial care to handoff to hospital to release and home care

Another problem identified by the inventors is related to patient self-care of the patient after hospital/clinic care. In many cases the patient drops off the map after an episode of care. The inventors have identified that there is no good mechanism for monitoring post care patient conditions and its additional goals of reducing readmissions and early diagnosis of post care issues based on monitoring.

Another related problem identified by the inventors is that tele-health should, but does not, offer post care monitoring of healthy lifestyle changes by a patient based on post care monitoring and trends based on post care data monitoring.

Another problem identified by the inventors also includes the ability of tele-health system that work with remote teams of consultants and care members in consulting on a remote patient from more than many locations simultaneously. More concise management of schedules, availability, and skill sets to uniquely address a particular client represents a very complex problem to solve.

Another problem identified by the inventors is the use of call centers to schedule and align consultants to a particular patients is very ponderous and difficult to optimize when a highly skilled consultant is desired for a consultation and offers few options to create consultation queues which would allow a particular provider to direct calls/consults to an immediately available consultant or to be queued for a particular consulting practice.

Another problem identified by the inventors is that a consultant in most tele-health instances is prevented from virtual “rounding” or visiting a number of similarly situated patients where a consultant can switch between consults virtually and give similar advisory or nuanced advisory depending on the related diagnosis requested.

Another problem identified by the inventors is that tele-health systems do not allow for the conditional triage of patients to the tele-health system where the more seriously ill patients to queue and alert the consultant to the more serious patient in queue and allow them to be promoted to the consultant for immediate review.

Another problem noticed is tele-health’s′ lack of auto-routing of patients to the right specialist. Of particular concern is for consultations where the patient is gravely ill or time is of the essence for treatment, it is particularly problematic if tele-health calls to consultants aren’t routed to the appropriate specialty and routed also shunted to a next consultant if the first choice is unavailable from a prioritized list of available consultants

Another problem detected in tele-health systems is that textual and audio transcriptions of each session are not auto scribed, parsed and indexed for notes, auto-population of future consults or future suggestions for a clinical decision support systems

And another problem noted by the inventors is a lack of continuous or continuity between episodes of care. The lack of this unity throughout a lifecycle of care for a patient may comprise: transitions from emergency response to treatment in an ambulance, to hospital care, to ambulatory care to home care all seem to contemplate a non-continuous care cycle.

Another problem identified is that patients post treatment vitals and recovery information is generally unavailable for monitoring.

Following on to this monitoring problem, the inventors have identified an additional problem with the collection and presentation of subjective patient data surveys/EMR/imaging data. This problem is further exacerbated by the lack of AI tools for the remote patient monitoring (RPM) that would assist both in hospital and outpatient services like rounding by remote physicians that can be trained to alert or enhance the patient monitoring to detect variances that would normally not be caught by lesser skilled physician and potentially suggest causes for the variances to care specialists or alert monitoring teams to enhanced risks based on the monitoring profile.

A problem further unaddressed in RPM is the analysis and detection of behavioral or psychological changes of patients in remote locations (home/clinics/Skilled Nursing Facilities (SNF) that would enhance a diagnosis. In many tele-health interchanges, the video/audio linkages to not support a full emotional psychological profile by the interviewer. Finally, the automation and capture of tele-health interview notes between a consulting physician and a patient is also largely unaddressed in these systems.

### BRIEF SUMMARY

The inventors have identified a solution that answers these problems as well as several more as disclosed in this specification. The solution centers around a unified platform concept that allows the actors in a care lifecycle for a particular patient are particularly well suited for a modularized tele-health system that connects in a variety of methodologies to a variety of mobile/clinical/hospital/managed care technical platforms.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

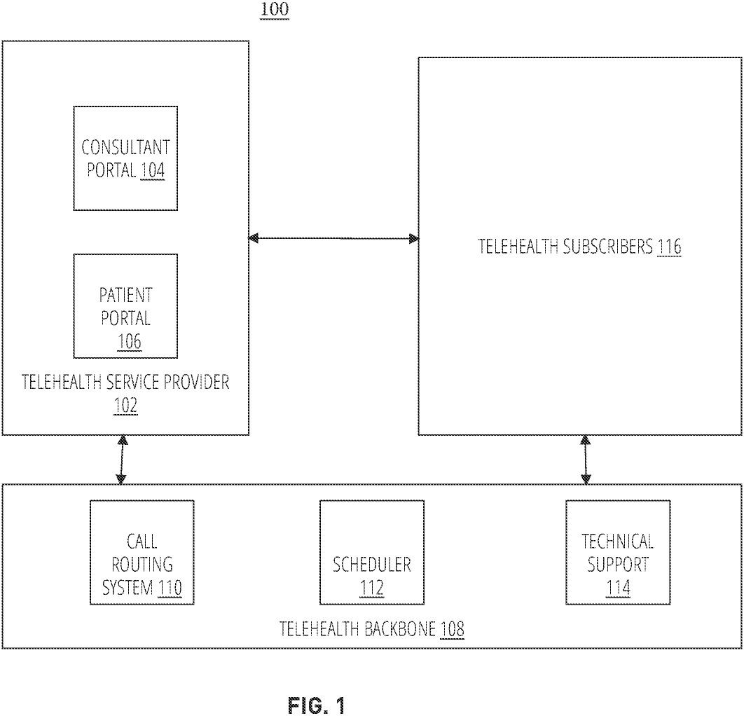

FIG. 1 illustrates a telehealth environment with aspects of the subject matter in accordance with one embodiment.

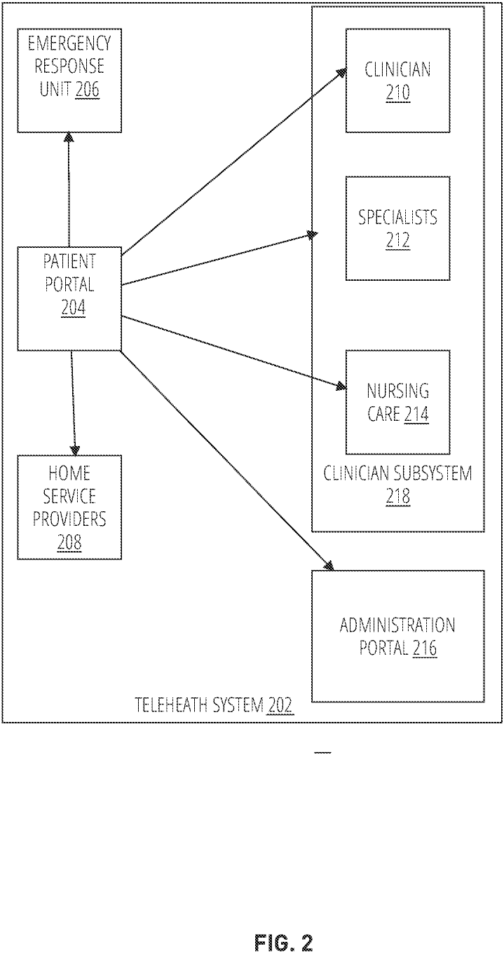

FIG. 2 illustrates a telehealth system with portals for various team and patient access in accordance with one embodiment.

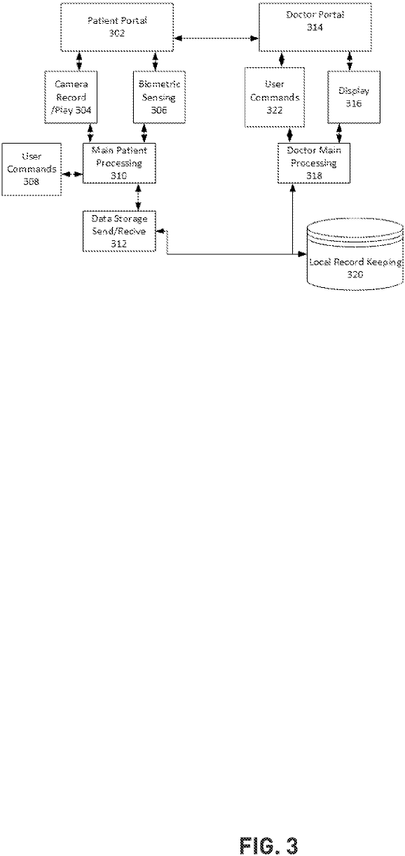

FIG. 3 illustrates a data flow during a tele-health consult in accordance with one embodiment.

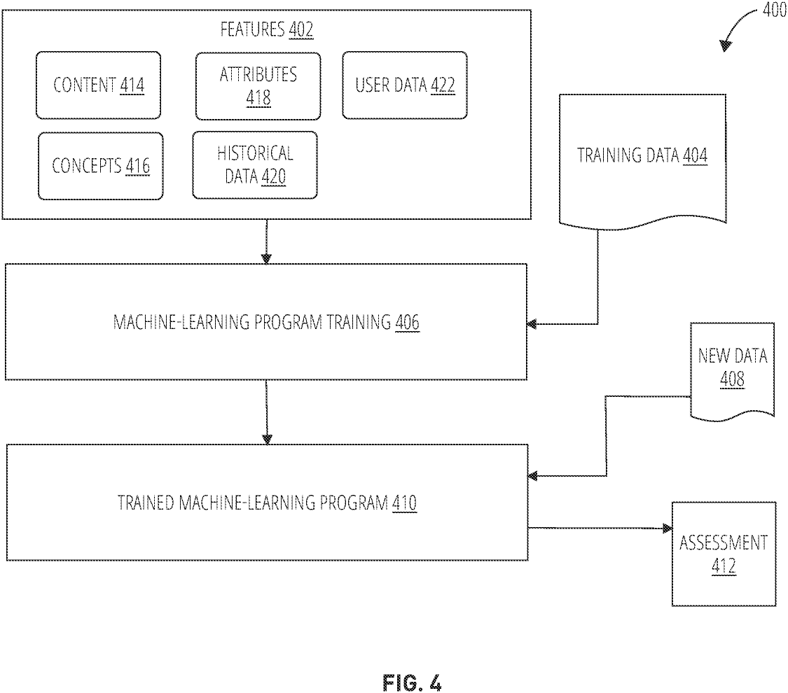

FIG. 4 illustrates training and use of a machine-learning program, according to some example embodiments.

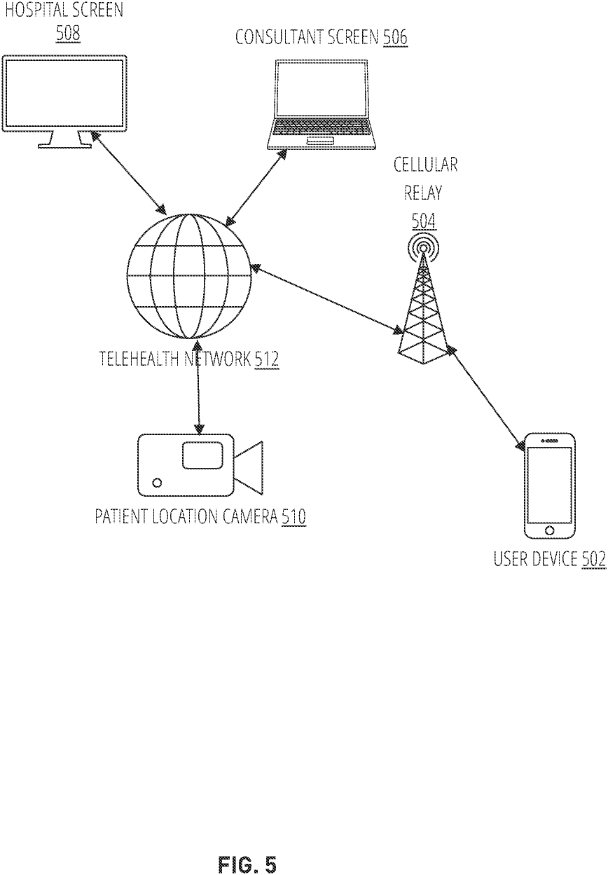

FIG. 5 illustrates minimal Tele-health System in accordance with one embodiment.

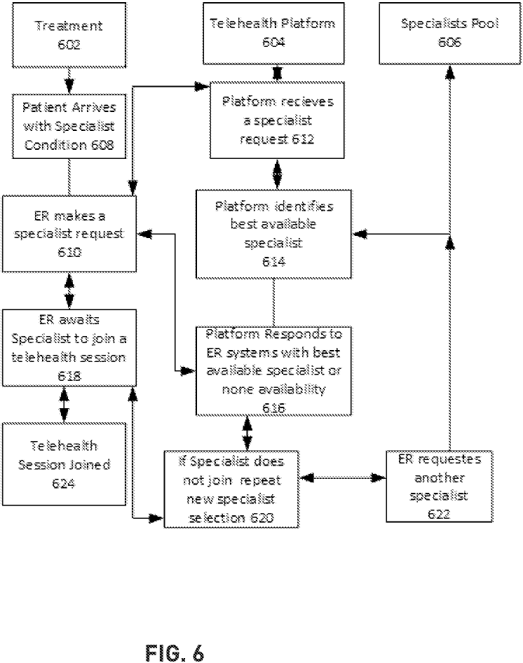

FIG. 6 illustrates a Tele-health System engagement in accordance with one embodiment.

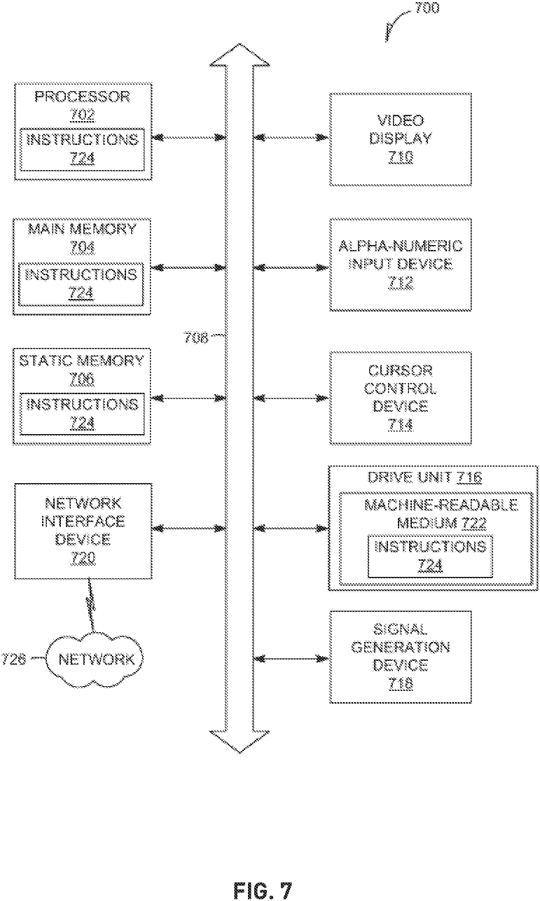

FIG. 7 illustrates a user machine of the subject matter in accordance with one embodiment.

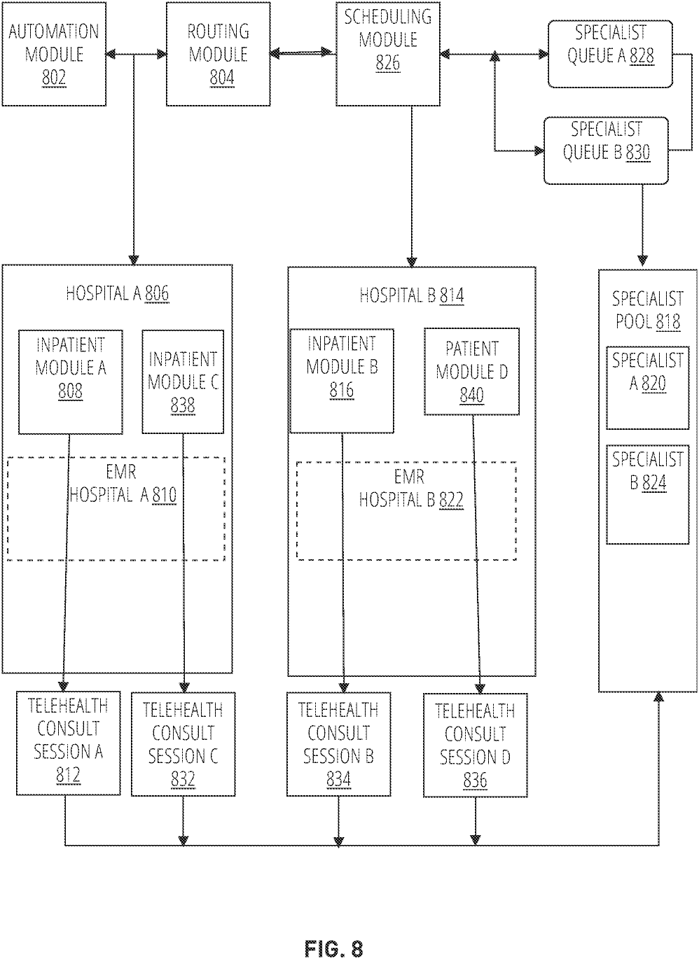

FIG. 8 illustrates and example of care continuum while scheduling specialists during a telehealth episode

### DETAILED DESCRIPTION

Embodiments of this solution may be implemented in one or a combination of hardware, firmware and software. Embodiments may also be implemented as instructions stored on a computer-readable storage device, which may be read and executed by at least one processor to perform the operations described herein. A computer-readable storage device may include any non-storing information in a form readable by a machine (e.g., a computer). For example, a computer-readable storage device may include read-only memory (ROM), random-access memory (RAM), magnetic disk storage media, optical storage media, flash-memory devices, cloud servers or other storage devices and media. Some embodiments may include one or more processors and may be configured with instructions stored on a computer-readable storage device. The following description and the referenced drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The first aspect of tele-health systems deficiencies solved by this solution is to allow connectivity between many of the surrounding systems from an emergency response system to the EHR systems to scheduling and availability systems of various consulting service personnel. By design the present solution using a number of standards based methodologies

Another one aspect of the solution identified by the inventors is that traditional hospital information modules (HIM) do not deal well with the rubric of the tele-health work stream namely integrating with a number of tele-health and EHR products while maintaining compliance with HIPAA and other privacy regimes that indirectly impact data sets related to PHI. The inventors solve this by maintaining strict data role across the system interfaces. The patient is de-identified in all situations were disclosure is not necessary, the API interfaces only link data fields necessary to track, assign, and respond to the main platform. The platform uses a set of variable entitlements assigned to various actors and systems that correspond to their level of data access and to their level of need for PII or PHI as appropriate.

Another problem identified with respect to tele-health-HIM systems is that alerts that would be seen in a large number of internal systems at hospitals are not seen by a tele-health consultant or in many cases do not exist in an ambulatory HIM. These missing or mistimed alerts can significantly alter a treatment regime if missed from a tele-health diagnosis. The solution disclosed here solves this problem at least two manners. The solution variants include access to Electronic Health Record (EHR) systems, public records, and other inferential systems that poll a number of systems where a patient’s record maybe supplemented if no data on their vitals, insurance or previous treatments is available from the primary EHR system. The solution uses disambiguation software to isolate, enhance and resolve missing data in the EHR files. This data enhancement could come from national records, employer records, health plan records, and other data copra.

A third problem identified with respect to the tele-health HIM is missing data from the various EHR sources that would normally be input by an attending nursing staff or medical assistant. This data in many cases is missed as the patient in some cases is out in the field being tended to by an EMT or in a smaller care facility where the respective attendants lack the training or the focus to input data correctly. The current solution feature uses a number of methods to first detect that there is missing data in fields that are normally populated from the various sending facilities, taking actions to update this information such as querying the facility to see if they have a paper record, querying other non-standard HIM modules to see if helpful data resides in non-standard systems, to allowing the data to be received from the patient or their relatives. Once the missing data is input, a separate Machine Learning module evaluates the prior data’s usefulness and likely accuracy. Additionally a Machine Learning algorithm flags outlier data sets as each patient EHR record is accessed to help improve treatment and assist each consultant reviewing a record in the correct context. Subordinate to this solution is the use of NLP tools to capture and digitize all conversations engaged between patient, care teams or attending physicians. These conversations can be indexed and annotated by either human review or by intelligence/machine learning AI/ML tools looking for patterns in the treatment, care, behavior, and outcomes of the care received. The NLP ingestion and processing of care interactions also can assist the subsequent diagnosis if a patient is returned to care post treatment.

Another problem identified is that tele-health takes the patient in a number of locations where full diagnostic or EHR data is missing for the tele-health HIM usage. Thus a problem of how to fill in or estimate missing data is also a substantial problem with the tele-health expert being shown a scene from an accident, a battlefield, an ambulance or other non-clinical settings presents itself to the tele-health expert. This current solution feature addresses this problem by several means. The solution a) asks an attending human for the data, b) asks machines/sensors attached to the patient for information or estimates data if possible given other parameters that fit a patient model in the EHR modeling. The estimated data is earmarked as estimated and will not enter the actual EHR unless approved and validated by a reviewing professional.

Another problem identified is that consultants maybe in remote sites as well limited by bandwidth or connectivity and a tele-health system struggles to provide the right information at the right time. The solution uses machine learning algorithms to model when information is optimally used by consultants and provided in a queued manner to allow a particular consultant to select the optimal information for their consultation and then retain that as a personalized profile preference. Meanwhile the data of selection will be utilized in a manner to set “best practices” configurations for data for a classification group of consultants. This solution will continuously optimize presentation of data and optimize other information like order sets, x-rays, and other diagnostic data to optimize the consultants workflow. This becomes even more important when the consultant is a highly sought after specialists. The current solution feature learns each specialist’s routine and pre-queues information based on previous similar consults. The solution even contemplates presenting training configurations based on a learned methodology by the industries top consultants to support and enhance diagnostic excellence of less trained or experience consultants

Another problem identified is that patients and consultants may both be in transitory or ambulatory conditions and a tele-health system struggles to adapt to this right information at the right times. This part of solution addresses a chronic aspect of healthcare problems in post hospital care. Healthcare generally is episodic at a hospital level with ongoing and lifestyle care left to clinics, personal physician care, and other healthy lifestyle consultants. This current solution feature comprises full lifecycle care in a manner that identifies patient behaviors, vital signs and other aspects of post episode care that would avoid re-admission. To this end the solution integrates with sensors that transition from the hospital care to a skilled nursing facility (SNF) to home care/monitoring to diminish any aspects of readmission that can be avoided. Often abbreviated as RPM (and sometimes known as remote patient management), remote patient monitoring is a method of healthcare delivery that uses the latest advances in information technology to gather patient data outside of traditional healthcare settings. RPM attempts to include physical, emotional and psychological health in a holistic view on remote patient care.

Behavioral health is also a very important component of tele-care in need of a solution. Emotional and Psychological modeling of remote patients is accomplished both by subjective interviews between patients and trained staff as well as by RPM video/audio/sensory data capture. This data capture would also include purely machine based detection of behavioral and psychological state of a patient by using facial recognition and voice samples, combining with subjective survey and past medical history data, utilizing artificial intelligence/machine learning (AI/ML) techniques to determine risks a patient can possess to themselves or others providing mechanism for early intervention by care team. Taking this a step further, a feature of the present solution would also use AI/ML “bots” These programs use artificial intelligence to deploy the principles of cognitive behavioral therapy and assist the care team in dealing with clinical and non-clinical forces on the patient’s recovery/care. As they are always available, the programs use natural language processing and learned responses to mimic conversation, remember past sessions and deliver advice around to the patient to engage with them when no human care provider is available. The bots can provide therapy and/or help calm patient if they are agitated or having suicidal thoughts. Various patient interaction with the bots can also alert care team if patient is at risk to themselves or others. Some of the behaviors identifiable by computer assisted behavioral therapy (CCBT) tools include challenging patient underlying assumptions of facts and irrational thoughts in treatment discussions, de-escalating suicidal thoughts, classifying how past experience can affect present feelings and beliefs, or helping the patient avoid generalizations and all-or-nothing thinking in diagnosis or recovery treatment.

Remote patient management is about using technologies to build the bridge that space between the traditional physical setting of healthcare, and where people really want to live every day. By using smartphones and tablets, the RPM system includes a RPM server for controlling the operation of the RPM system. The server has a processor a memory and a communication interface. The RPM server is coupled with a data store, which can store data generated and/or received by the server. The data store may also store one or more databases with various hardware and/or patient specific information. The server may generate data relating to site, floor, camera, and IP info. The server communicates with a RPM client, which is a client application that is used by a technician (e.g., user, observer, tele-monitor, or operator) for remote observation and communication from a remote station to one of the patient rooms that has a server end point that are organized as subnets. For example, the server communicates with subnets. For the RPM client may be operated on a desktop computer or another suitable computing device such as a specialized monitoring machine, but other devices depending on the type of monitoring desired laptop, a tablet or a smart phone.

The server is implemented as a server for camera discovery across different subnets on a network. The subnets may be implemented as Virtual Local Area Networks (VLANs). The subnets comprises a networked monitoring device and cameras. The subnet communicates with the server through the networked monitoring device.

The network may be a simple network (e.g., a single network with one type of IP), may be a complex network (e.g.. both wired/wireless networks, different subnets, multiple VLANs, multiple sites, different visibility, dynamic IP, and static IP), or in between. For example, the network may be all wired, all wireless, or both wired and wireless. The network may include wired subnets, wireless subnets, or both wired and wireless subnets 14. Similarly, each subnet 14 may be all wired, all wireless, or both wired and wireless. Also, for example, a portion or all of the network may use dynamic IP, static IP, or both. Similarly, each subnet 14 may use dynamic IP, static IP, or both.

Monitoring includes vital monitoring with attached sensors that allow a recovering patient as much mobility as possible. To this end, wearable sensors and devices are optimized to track a post episode patient as they regain their normal lifestyle. By example a patient recovering from cardiac bypass surgery may have monitoring of blood properties, respiratory properties coupled with pulse and pressure monitory using exercise devices. There may be daily ECG readings made by adherable sensors read by a monitoring app on their phone. There may be other implanted sensors that report the condition and chemistry of the blood in real time.

Another problem identified is the ability of patient handoff and coverage between care providers, ambulance services, EMTs and to successfully transfer all related matters between systems to from initial care to handoff to hospital to release and home care. The solution creates an episodic record that is handed of from one actor to the next actor. By example, a patient discovered unresponsive would have an episode record initiated by a first responder, the patient record could be initiated by a photo of the patient which might be correlated to a vision recognition profile of that person; alternatively a fingerprint would serve as an identifying attribute. The patient once identified would allow the solution to alert a first responder to any know conditions, allergies, heart disease, diabetes; which might indicate what is wrong with the patient. Also the first responder may be guided away from administering medication that might interact with a known medication that the patient might be experiencing. Alternatively, the first responders might also be alerted to any nearby family or dependents of the patient to allow other responders to find, assist and notify the others of the patient’s condition and to which hospital they may be transported to. Also at this time a consultant, may be brought online to suggest at the scene treatment based on the patient’s vital signs to help stabilize the patient to be transported to the hospital. Once the patient is ready to be transported, the solution hands off data to the ambulance systems which continues to allow a consultant to recommend treatment during the ambulance ride. Once a patient is delivered to the emergency room of the hospital, the patient is handed off from the ambulance system to the hospital systems with a full transfer of all details know to that point over to the hospital system.

Another problem identified by the inventors is related to patient self-care of the patient after hospital/clinic care. In many cases the patient drops off the map after an episode of care and is left to their own care with a family member holding a packet of care instructions. In some cases, the patient is either confused or neglects to execute those care instructions. This results in a readmission as the patient fails to recover in post care mode. The inventors have identified that there is no good mechanism for monitoring post care patient conditions and its additional goals of reducing readmissions and early diagnosis of post care issues based on monitoring. The solution as disclosed provides tele-health monitoring, trend capture, routine evaluation and allows for a consult with a doctor, nurse, social worker, or psychologist as needed without requiring a readmission to the hospital. Additional monitoring could include diet, exercise, hygiene, and social skills recommendations also without re-entering the hospital. The goal of this solution variant would be to provide daily notice, reaffirmation and engagement with the patient to keep mood, physical health, and family support aligned to an optimal recovery. The solution enhances this work stream by the addition of various applied sensors that allow more sophisticated monitoring of vitals and other trending conditions related to blood chemistry, pharmaceutical efficacy and other clinical questions that the patient’s EHR might suggest in light of those elements.

The solution as disclosed also offers post care monitoring of healthy lifestyle changes by a patient based on post care monitoring and trends based on post care data monitoring. This includes aligning the patient with nutritionists, dietitians, psychologists, and other non-physician counseling.

Another problem identified by the inventors also includes the ability of tele-health system that work with remote teams of consultants and care members in consulting on a remote patient from more than many locations simultaneously. More concisely management of schedules, availability, and skill sets to uniquely address a particular client represents a very complex problem to solve. The solution contemplated uses consulting resumes, geolocation data, scheduling data and other profiling data to review, organize, prioritize, sets of remotely located multiple individuals, teams, equipment and time zones to orchestrate a multitude of consulting engagement. This is further compounded by widely varying compensation structure for treatment, insurance, medical licensing that may reach across, state, national and multinational borders The inventors of this solution utilize automated services to “game” optimal schedules that optimize consultants work flow in their natural time zone.

Another aspect of this solution is to automate scheduling and eliminating the use of human call centers to schedule and align consultants to a particular patient. The current solution schedules, multiple scheduling solutions and polls consultants schedules and allows various consultants to “bid” for their optimized schedules and then allow the tele-health system to backfill the schedule with the next available consultant..

Another solution facet identified by the inventors is that a consultant in most tele-health instances is prevented from virtual “rounding” or visiting a number of similarly situated patients where a consultant can switch between consults virtually and give similar advisory or nuanced advisory depending on the related diagnosis requested. The solution can put the Another problem identified by the inventors is that tele-health systems do not allow for the conditional triage of patients to the tele-health system where the more seriously ill patients to queue and alert the consultant to the more serious patient in queue and allow them to be promoted to the consultant for immediate review.

Another problem noticed is tele-health’s′ lack of auto-routing of patients to the right specialist. As each telehealth call is routed to the appropriate specialty and the call routed to a prioritized list of available consultants. The current solution has a faceted voting mechanism using ML to assist the routing of patients to the best available consultant rather than a long queue for a specific consultant.

Another problem detected in tele-health systems is that textual and audio transcriptions of each session are not auto scribed, parsed and indexed for notes, auto-population of future consults or future suggestions for a clinical decision support systems

And another problem noted by the inventors is a lack of continuous or continuity between episodes of care. The lack of this unity throughout a lifecycle of care for a patient may comprise: transitions from emergency response to treatment in an ambulance, to hospital care, to ambulatory care to home care all seem to contemplate a non-continuous care cycle.

In aspect of the current solution, a patient- consultant tele-health system, comprises:
1. at least one article of medical equipment for purposes of recording a patient attribute at a patient location;
2. a remote recording system for use at the patient location, comprising the capture of audio data via an input device and/or the capture of video data via a video camera;
3. a video output device; and an audio output device at the location of a consultant;
   wherein at least one of the audio input device and the video camera are adapted to accept a first communication from a first user of the patient-consultant tele-health system at the patient location for transmission to a second user at a consultant’s location remote from the patient location, and wherein at least one of the video output device and the audio output device are adapted to include a shared communication of the data to another consultant;

A further feature of the solution comprises electrical control circuitry including circuitry for receiving a medical equipment data signal from the at least one article of medical equipment, the medical equipment data signal including medical data acquired with the at least one article of medical equipment; or an operational mode determination module configured to determine a consultant’s availability using the tele-health system’s availability including from at least two consultants are available modes of the patient-consultant tele-health system, wherein the consulting mode of the patient-consultant tele-health system includes at least one of one or more consulting attributes of the at least one article of medical equipment or one or more operational mode of the remote viewing system; and a queuing module configured to make ready all relative data of the patient for a consultation as well as queuing other video and audio artifacts from other patient events of the patient-consultant tele-health system in the consultant tele-health system operational mode;

and communication circuitry for transmitting the consultant tele-health system queuing data signal and an identification data signal indicative of at least one of a session readiness of at least a portion of the patient-consultant tele-health system or an identity of the first user of the patient-consultant tele-health system to the consultant’s location; and communicating information between the electrical control circuitry at the patient location and the consultant’s location, the information including at least one of the medical data acquired with the at least one article of medical equipment, at least one instruction for controlling the at least one article of medical equipment, or a remote viewing system communication signal including at least one of the first communication and the second communication.

In a solution feature, a method of controlling a patient to consultant tele-health system comprising accepting a first communication from a first user of the patient-consultant tele-health system at a patient location via a remote viewing system at the patient location, the remote viewing system including an audio input device, a video camera, a video output device, and an audio output device, and wherein the patient-consultant tele-health system is located at the patient location and includes the remote viewing system, at least one article of medical equipment, a user identity input device, communication circuitry, and electrical control circuitry; transmitting the first communication to a consultant’s location via the communication circuitry; receiving a second communication from the consultant’s location with the communication circuitry; presenting the second communication to the first user via the remote viewing system; receiving a signal from the at least one an article of medical equipment with the electrical control circuitry, the signal including medical data acquired with the at least one article of medical equipment; determining an operational mode data signal, wherein the operational mode data signal is indicative of at least one operational mode of the at least one article of medical equipment; determining a queuing data signal, wherein the queuing data signal is indicative of an readiness for viewing of the at least one article of medical equipment in the at least one operational mode, and wherein the queuing data signal includes data representing data of a clinical consultation of a patient further comprising of the at least one article of medical equipment in the at least one operational mode; transmitting the operational mode data signal to the consultant’s location; transmitting the queuing data signal to the consultant’s location; transmitting an identification data signal indicative of at least one of a session readiness of at least a portion of the patient-consultant tele-health system or an identity of the first user of the patient-consultant tele-health system to the consultant’s location; and communicating information between the electrical control circuitry at the patient location and the consultant’s location via the communication circuitry at the patient location, the information including at least one of the medical data acquired with the at least one article of medical equipment or at least one instruction for controlling the at least one article of medical equipment. In addition to the foregoing, other method aspects are described in the claims, drawings, and text forming a part of the disclosure set forth herein.

In a solution feature, an article of manufacture comprises one or more non-transitory machine-readable data storage media bearing one or more instructions for accepting a first communication from a first user of the patient-consultant tele-health system at a patient location via a remote viewing system at the patient location, the remote viewing system including an audio input device, a video camera, a video output device, and an audio output device, and wherein the patient-consultant tele-health system is located at the patient location and includes the remote viewing system, at least one article of medical equipment, a user identity input device, communication circuitry, and electrical control circuitry; transmitting the first communication to a consultant’s location via the communication circuitry; receiving a second communication from the consultant’s location with the communication circuitry; presenting the second communication to the first user via the remote viewing system; receiving a signal from the at least one an article of medical equipment with the electrical control circuitry, the signal including medical data acquired with the at least one article of medical equipment; determining an operational mode data signal, wherein the operational mode data signal is indicative of at least one operational mode of the at least one article of medical equipment; determining a queuing data signal, wherein the queuing data signal is indicative of an aggregated date related to a patient further comprising of the at least one article of medical equipment in the at least one operational mode, wherein the queuing data signal also includes the electronic health record and the at least one article of medical equipment in the at least one operational mode; transmitting the operational mode data signal to the consultant’s location; transmitting the queuing data signal to the consultant’s location; transmitting an identification data signal indicative of at least one of a session readiness of at least a portion of the patient-consultant tele-health system or an identity of the first user of the patient-consultant tele-health system to the consultant’s location; and communicating information between the electrical control circuitry at the patient location and the consultant’s location via the communication circuitry at the patient location, the information including at least one of the medical data acquired with the at least one article of medical equipment, or at least one instruction for controlling the at least one article of medical equipment. In addition to the foregoing, other aspects are of such an article of manufacture are described in the claims, drawings, and text forming a part of the disclosure set forth herein.

In a solution feature, a method of providing consultative visual or audio monitoring of a subject comprises, when a remote visualization system is at a first location, receiving at a second location remote from the first location a first image of at least a portion of a subject from communication circuitry of the remote visualization system, wherein the first image was captured at a first lighting condition with an imaging system of the remote visualization system, wherein the first image includes at least one feature, and wherein the remote visualization system includes an audio input device, the imaging system, a video output device, an audio output device, a controllable lighting system including at least one light source, the communication circuitry, and electrical control circuitry configured to control operation of the audio input device, imaging system, video output device, audio output device, and controllable lighting system; detecting the at least one feature of the first image with image processing circuitry at the second location, the image processing circuitry including at least one of image processing hardware and software; determining with lighting parameter control circuitry at the second location an adjustment to the controllable lighting system based at least in part on the at least one detected feature, wherein the adjustment to the controllable lighting system is determined to modify an amount or type of medically useful information in the image based upon analysis of information content of the image determined from the at least one detected feature; determining with the lighting parameter control circuitry at the second location a lighting control signal based at least in part on the determined adjustment to the controllable lighting system; when the remote visualization system is at the first location, transmitting the lighting control signal from the second location to the first location for receipt by the communication circuitry for controlling the adjustment to the controllable lighting system to provide a second lighting condition at the first location; and receiving at the second location a second image of the at least a portion of the patient from the communication circuitry, wherein the second image was captured at the second lighting condition with the imaging system; wherein at least one of the first image and the second image contains information indicative of a health status of the patient, and wherein the adjustment to the controllable lighting system influences at least one of the amount or type of medically useful information indicative of the health status of the patient in the second image of the patient. In addition to the foregoing, other method aspects are described in the claims, drawings, and text forming a part of the disclosure set forth herein.

In a solution feature, a remote visualization system comprises, an audio input device; an imaging system adapted to acquire an image of a subject, the image containing information indicative of a health status of the patient; a video output device; an audio output device;, and at least one controllable positioning system configured to adjust at least one of position, orientation, or aiming of the at least one light source; first electrical control circuitry operatively connected to and configured to control operation of the audio input device, imaging system, video output device, audio output device, and controllable lighting system, wherein the first electrical control circuitry includes timing circuitry; and communication circuitry configured to, when the remote visualization system is at a first location, provide communication between the first electrical control circuitry of the remote visualization system and second electrical control circuitry at a second location remote from the first location and to receive a lighting control signal from the second electrical control circuitry; wherein the first electrical control circuitry is configured to control the controllable lighting system in response to the lighting control signal, wherein controlling the controllable lighting system includes at least one of controlling the controllable positioning system to adjust at least one of the position, the orientation, or the aiming of the at least one light source of the controllable lighting system or controlling the at least one controllable parameter of the light pulse to increase at least one of an amount or type of medically useful information in an acquired image of the patient. In addition to the foregoing, other system aspects are described in the claims, drawings, and text forming a part of the disclosure set forth herein.

In a solution feature, an article of manufacture comprises, one or more non-transitory machine-readable data storage media bearing one or more instructions for, when a remote visualization system is at a first location, receiving at a second location remote from the first location a first image of at least a portion of a subject from communication circuitry of the remote visualization system, wherein the first image was captured at a first lighting condition with an imaging system of the remote visualization system, wherein the first image includes at least one feature, and wherein the remote visualization system includes an audio input device, the imaging system, a video output device, an audio output device, a controllable lighting system including at least one light source, the communication circuitry, and electrical control circuitry configured to control operation of the audio input device, imaging system, video output device, audio output device, and controllable lighting system; one or more instructions for detecting the at least one feature of the first image with image processing circuitry at the second location, the image processing circuitry including at least one of image processing hardware and software; one or more instructions for determining with lighting parameter control circuitry at the second location an adjustment to the controllable lighting system based at least in part on the at least one detected feature, wherein the adjustment to the controllable lighting system is determined to modify an amount or type of medically useful information in the image based upon analysis of information content of the image determined from the at least one detected feature; one or more instructions for determining with the lighting parameter control circuitry at the second location a lighting control signal based at least in part on the determined adjustment to the controllable lighting system; one or more instructions for, when the remote visualization system is at the first location, transmitting the lighting control signal from the second location to the first location for receipt by the communication circuitry for controlling the adjustment to the controllable lighting system to provide a second lighting condition at the first location; and one or more instructions for receiving at the second location a second image of the at least a portion of the patient from the communication circuitry, wherein the second image was captured at the second lighting condition with the imaging system; wherein at least one of the first image and the second image contains information indicative of a health status of the patient, and wherein the adjustment to the controllable lighting system influences at least one of the amount or type of medically useful information indicative of the health status of the patient in the second image of the patient. In addition to the foregoing, other aspects are of such an article of manufacture are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

Embodiments of this solution may be implemented in one or a combination of hardware, firmware and software. Embodiments may also be implemented as instructions stored on a computer-readable storage device, which may be read and executed by at least one processor to perform the operations described herein. A computer-readable storage device may include any non-storing information in a form readable by a machine (e.g., a computer). For example, a computer-readable storage device may include read-only memory (ROM), random-access memory (RAM), magnetic disk storage media, optical storage media, flash-memory devices, cloud servers or other storage devices and media.

Some embodiments may include one or more processors and may be configured with instructions stored on a computer-readable storage device. The following description and the referenced drawings sufficiently illustrate specific embodiments to enable those skilled in the art to practice them.

Other embodiments may incorporate structural, logical, electrical, process, and other changes. Portions and features of some embodiments may be included in, or substituted for, those of other embodiments. Embodiments set forth in the claims encompass all available equivalents of those claims.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

With reference to FIG. 1, an environment 100 in which example embodiments of the inventive subject matter may be practiced is shown. The environment 100 comprises a Tele-health Backbone 108 in communication with both a patient subsystem and a Consultant Portal 104. The Tele-health Backbone 108 provides an Administration Module to route Patient Portal s 204 to appropriate consultant organizations.

With Reference to FIG. 2. The Teleheath System 202 comprises a set of portals, a Consultant Portal 104 serving those consultants offering tele-health services, a Patient Portal 106 where the Patient Portal 204 where the patient or a representative of the patient can manage their interactions, an Emergency Response Unit 206 portal where an EMT or some other first responder can interact with the patients data

The various components of the environment may be communicatively coupled together through one or more networks. The networks may comprise, for example, one or more of a wired or wireless network, a local area network (LAN), or a wide area network (WAN).

FIG. 3 illustrates the data flow during a typical tele-health consult. This figure shows the data flow in both clinical and non-clinical settings between the patient module 302 and the doctor portal 314 Data from camera 304, biosensor sensing 306 flows to the 320 Main Patient processing, based on user commands 308 processing and storing and sending data 312 between the Patient Portal and the Doctor Portal 314 allowing the Doctor to access, Display 316 and command 322 and store relevant telehealth data in Local Storage

FIG. 4 illustrates training and use of a machine-learning program (MLP) 400, according to some examples of the present solution. In tele-health, the use of machine-learning programs (MLPs), also referred to as machine-learning algorithms or tools, are used to perform operations associated with tele-health AI being used to accomplish many critical process comprising diagnosis, clinical order sets, insurance actions, voice transcription among other important tasks as described herein. Machine learning explores the study and construction of algorithms, also referred to herein as tools that may learn from existing data and make predictions about new data. Such machine-learning tools operate by building a model from example training data 404 in order to make data-driven predictions or decisions expressed as outputs or tele-health assessments (e.g., assessment 412). Although example embodiments are presented with respect to a few machine-learning tools, the principles presented herein may be applied to other machine-learning tools especially when tele-health.

In some example embodiments, different machine-learning tools may be used. For example, Logistic Regression (LR), Naive-Bayes, Random Forest (RF), neural networks (NN), matrix factorization, and Support Vector Machines (SVM) tools may be used for classifying or scoring job postings.

Two common types of problems in machine learning are classification problems and regression problems. Classification problems, also referred to as categorization problems, aim at classifying items into one of several category values (for example, is this object an apple or an orange?). Regression algorithms aim at quantifying some items (for example, by providing a value that is a real number).

The machine-learning algorithms use features 402 for analyzing the data to generate an assessment 412 to discover. Each of the features 402 is an individual measurable property of a phenomenon being observed (e.g., drug efficacy or other measurable treatment phenomenon. The concept of a feature is related to that of an explanatory variable used in statistical techniques such as linear regression. Choosing informative, discriminating, and independent features is important for the effective operation of the MLP in pattern recognition, classification, and regression. Features may be of different types, such as numeric features, strings, and graphs.

In one example embodiment, the features 402 may be of different types and may include one or more of content 414, concepts 416, attributes 418. historical data 420 and/or user data 422, merely for example.

The machine-learning algorithms that apply to the routing data use the training data 404 to find correlations among the identified features 402 that affect the outcome or assessment 412. In some example embodiments, the training data 404 includes labeled data, which is known data for one or more identified features 402 and one or more outcomes, such as detecting communication patterns, detecting the meaning of the message, generating a summary of a message, detecting action items in messages detecting urgency in the message, detecting a relationship of the user to the sender, calculating score attributes, calculating message scores, etc.

With the training data 404 and the identified features 402, the machine-learning tool is trained at machine-learning program training 406. The machine-learning tool appraises the value of the features 402 as they correlate to the training data 404. The result of the training is the trained machine-learning program 410.

In the case of telehealth there is an additional challenge as the algorithms are also tasked with identifying and sequestering PII and in presenting the data to the system. When the trained machine-learning program 410 is used to perform an assessment, new data 408is provided as an input to the trained machine-learning program 410, and the trained machine-learning program 410 generates the assessment 412 as output.

With regard to FIG. 5, it shows a minimal Tele-health System 202 set up where a mobile User Device 502 or a Patient Location camera 510 can be set up to initiate a Tele-health network 512 session. The Tele-health networks 512 comprises a Tele-health Backbone 108, Tele-health Subscribers 116 and Consultant Portals 104. The patient may be observed using either via a User Device 502 or a dedicated Patient Location camera 510 from an EMT or other Emergency Response Unit 206, the Tele-health networks 512 identified available Consultant Screen 506 or Hospital Screen 508 and creates a patient episode for a treatment. If the patient has been treated earlier, relevant data is extracted from the EHR and made available to the right consultant tasked with care.

FIG. 6 describes an example of a Tele-health Systems as a typical solution engagement. The Figure describes a ER care situation which treatment 602 where a specialist is required. The telehealth platform 604 uses its Machine Learning to select the correct specialist depending on the comorbidities of the patient, the available ER staff, and the roster of specialists 606. The ER makes a request 608 and the telehealth platform 604 receives a request 612 and recommends a specialist based on availability and skill set of the specialist. The ER awaits the specialist to join 618, and if the specialist joins the telehealth session, it proceeds without further delay. In the event of a non-joinder, or none available status, the telehealth will then attempt to find the next best specialists defined by a number of data factors 620.

With reference to FIG. 7, an embodiment of a user machine 700 within which instructions for causing the machine to perform any one or more of the methodologies discussed herein may be executed. In alternative example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a Personal Digital Assistant (PDA), a cellular telephone, a web appliance, a network router, a switch or bridge, a server, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 700 may include a processor 702 (e.g., a central processing unit (CPU), a graphics processing unit (GPU) or both), a main memory 704 and a static memory 706, which communicate with each other via a bus 708. The computer system 700 may further include a video display unit 710 (e.g., a liquid crystal display (LCD) or a cathode ray tube (CRT)). In example embodiments, the computer system 700 also includes one or more of an alpha-numeric input device 712 (e.g.. a keyboard), a user interface (UI) navigation device or cursor control device 714 (e.g., a mouse), a disk drive unit 716. a signal generation device 718 (e.g., a speaker), and a network interface device 720.

The disk drive unit 716 includes a machine-readable storage medium 722 on which is stored one or more sets of instructions 724 and data structures (e.g., software instructions) embodying or used by any one or more of the methodologies or functions described herein. The instructions 724 may also reside, completely or at least partially, within the main memory 704 or within the processor 702 during execution thereof by the computer system 700, the main memory 704 and the processor 702 also constituting machine-readable media.

While the machine-readable storage medium 722 is shown in an exemplary embodiment to be a single medium, the term “machine-readable storage medium” may include a single storage medium or multiple media (e.g., a centralized or distributed database, or associated caches and servers) that store the one or more instructions 724. The term “machine-readable storage medium” shall also be taken to include any tangible medium that is capable of storing, encoding, or carrying instructions for execution by the machine and that causes the machine to perform any one or more of the methodologies of embodiments of the present invention, or that is capable of storing, encoding, or carrying data structures used by or associated with such instructions. The term “machine-readable storage medium” shall accordingly be taken to include, but not be limited to, solid-state memories, optical and magnetic media, and non-transitory machine-readable storage media. Specific examples of machine-readable storage media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and CD-ROM and DVD-ROM disks.

The instructions 724 may further be transmitted or received over a communications network 726 using a transmission medium via the network interface device 720 and utilizing any one of a number of well-known transfer protocols (e.g., HTTP). Examples of communication networks include a local area network (LAN), a wide area network (WAN), the Internet, mobile telephone networks, Plain Old Telephone (POTS) networks, and wireless data networks (e.g.. WiFi and WiMax networks). The term “transmission medium” shall be taken to include any intangible medium that is capable of storing, encoding, or carrying instructions for execution by the machine, and includes digital or analog communications signals or other intangible medium to facilitate communication of such software. FIG. 8 describes an automated call routing feature of the solution. This routing and scheduling modules organize the queues related to specific specialists and prepares all necessary information from the consulted patients EHR and works through privacy and security screens to only display information that is authorized to a particular viewer. This

Certain embodiments described herein may be implemented as logic or a number of modules, engines, components, or mechanisms. A module, engine, logic, component, or mechanism (collectively referred to as a “module”) may be a tangible unit capable of performing certain operations and configured or arranged in a certain manner. In certain exemplary embodiments, one or more computer systems (e.g., a standalone, client, or server computer system) or one or more components of a computer system (e.g., a processor or a group of processors) may be configured by software (e.g., an application or application portion) or firmware (note that software and firmware can generally be used interchangeably herein as is known by a skilled artisan) as a module that operates to perform certain operations described herein.

In various embodiments, a module may be implemented mechanically or electronically. For example, a module may comprise dedicated circuitry or logic that is permanently configured (e.g., within a special-purpose processor, application specific integrated circuit (ASIC), or array) to perform certain operations. A module may also comprise programmable logic or circuitry (e.g., as encompassed within a general-purpose processor or other programmable processor) that is temporarily configured by software or firmware to perform certain operations. It will be appreciated that a decision to implement a module mechanically, in the dedicated and permanently configured circuitry, or in temporarily configured circuitry (e.g., configured by software) may be driven by, for example, cost, time, energy-usage, and package size considerations.

Accordingly, the “module” should be understood to encompass a tangible entity, be that an entity that is physically constructed, permanently configured (e.g.. hardwired), or temporarily configured (e.g., programmed) to operate in a certain manner or to perform certain operations described herein. Considering embodiments in which modules or components are temporarily configured (e.g., programmed), each of the modules or components need not be configured or instantiated at any one instance in time. For example, where the modules or components comprise a general-purpose processor configured using software, the general-purpose processor may be configured as respective different modules at different times. Software may accordingly configure the processor to constitute a particular module at one instance of time and to constitute a different module at a different instance of time.

Modules can provide information to, and receive information from, other modules. Accordingly, the described modules may be regarded as being communicatively coupled. Where multiples of such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory device to which it is communicatively coupled. A further module may then, at a later time, access the memory device to retrieve and process the stored output. Modules may also initiate communications with input or output devices and can operate on a resource (e.g., a collection of information).

Although an overview of the inventive subject matter has been described with reference to specific exemplary embodiments, various modifications and changes may be made to these embodiments without departing from the broader spirit and scope of embodiments of the present invention. Such embodiments of the inventive subject matter may be referred to herein, individually or collectively, by the term “invention” merely for convenience and without intending to voluntarily limit the scope of this application to any single invention or inventive concept if more than one is, in fact, disclosed.

As used herein, the term “or” may be construed in either an inclusive or exclusive sense. Additionally, although various example embodiments discussed focus on a specific network-based environment, the embodiments are given merely for clarity in disclosure. Thus, any type of electronic system, including various system architectures, may employ various embodiments of the search system described herein and is considered as being within a scope of example embodiments.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present invention. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present invention as represented by the appended claims. The specification and drawings are, accordingly, to be regarded in an illustrative rather than a restrictive sense. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g.. one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as micro-code, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”. “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A tele-health system comprising:
a tele-health subsystem further comprising a plurality of portals having a plurality of functionalities;
wherein an individual portal from the plurality is unique to a particular actor in a continuum of care, the portal further comprising visual, auditory, textual and derived data presented in a controlled environment to enable a tele-health session;
a patient located tele-health subsystem further comprising audio and video interchange between at least two locations; wherein the audio and video interchange is a treatment episode related to a patient generated at the patient portal;
a consultant located tele-health subsystem further comprising audio and video interchange between at least two locations; wherein the audio and video interchange is a treatment episode related to a patient presented via a consultant portal; and
an administration tele-health subsystem further comprising an administration of a tele-health episode related to a patient that manages a set of all related data collected and presented regarding a particular patient during care and further comprising a data update function that adds a set of new data upon new information about the patient generated by at least one of the consultants.

2. The system of claim 1 further comprising a machine language algorithm that matches the consultant with factors selected from two or more comorbidities of the patient.

3. The system of claim 2 further comprising a machine language algorithm that matches the consultant with factors selected from two or more factors of the consultants availability.

4. The system of claim 3 further comprising a machine language algorithm that matches the consultant with factors selected from two or more availabilities of the consultant with a patients prior health record pushed out to the attending consultant.
